(19) United States
(12) Patent Application Publication
(10) Pub. No.: US 2026/0337617 A1
(43) Pub. Date: Oct. 29, 2026

(54) PROTEIN-NUCLEIC ACID COMPLEX, AND TARGET SUBSTANCE DETECTION KIT AND DETECTION METHOD USING SAME

(71) Applicant: NATIONAL UNIVERSITY CORPORATION TOKYO UNIVERSITY OF AGRICULTURE AND TECHNOLOGY, Fuchu-shi, Tokyo (JP)

(72) Inventors: Ryutaro ASANO, Fuchu-shi, Tokyo (JP); Kazunori IKEBUKURO, Fuchu-shi, Tokyo (JP)

(73) Assignee: NATIONAL UNIVERSITY CORPORATION TOKYO UNIVERSITY OF AGRICULTURE AND TECHNOLOGY, Fuchu-shi, Tokyo (JP)

(21) Appl. No.: 19/101,695

(22) PCT Filed: Aug. 29, 2023
(86) PCT No.: PCT/JP2023/031273

§ 371 Date: Feb. 6, 2025

(30) Foreign Application Priority Data
Aug. 31, 2022 (JP) 2022-138531

(51) Int. Cl. C12N 15/115; C12N 9/24

(52) U.S. Cl. CPC C12N 15/115; C12N 9/2497; C12Y 302/02027; C07K 2319/20; C12N 2310/16; C12N 2310/3513; C12N 2320/00

(57) ABSTRACT

Provided is a highly stable and versatile protein-nucleic acid complex. This protein-nucleic acid complex includes a nucleic acid molecule having uracil, a uracil DNA glycosylase domain covalently bound to the nucleic acid molecule, and a protein domain covalently bound directly or indirectly to the uracil DNA glycosylase domain.

### TECHNICAL FIELD

The present invention relates to a protein-nucleic acid complex having a nucleic acid molecule and a protein covalently bound to each other, a kit and method for detecting a substance using the protein-nucleic acid complex, and a method for using the protein-nucleic acid complex.

### BACKGROUND ART

In a complex of a nucleic acid and a protein, connecting the nucleic acid and the protein by a covalent bond is a big advantage. For example, when the complex is used as a sensing element, if association by a non-covalent bond is present, it is concerned that the bond may dissociate in a washing step of a complex synthesis process or a system using the complex. The DNA-covalently binding proteins presently reported are roughly classified into a HUH endonuclease (histidine-hydrophobic residue-histidine-endonuclease) group and a uracil DNA glycosylase (uracil DNA glycosylase X: UdgX) group.

Of them, UdgX is described in Patent Literature 1 to form a complex with a nucleic acid. Patent Literature 1 discloses that when an antibody-binding protein such as protein A is fused to UdgX, a complex of an antibody, UdgX and a nucleic acid bound to UdgX can be formed. Patent Literature 1 discloses that the complex can be formed by using a nucleic acid molecule comprising uracil as a probe and binding it to UdgX and used in immuno PCR. UdgX derived from Mycobacterium smegmatis is an enzyme belonging to the uracil-DNA glucosidase family and involved in repairing a mismatch of nucleotide sequence on a nucleic acid. UdgX is an enzyme which recognizes uracil of ssDNA (single-stranded DNA) or double-stranded DNA (dsDNA) and removes it, and then, forms a glycosidic bond at the abasic site via a histidine residue (Non Patent Literature 1).

In the meantime, a complex of a nucleic acid and a protein can be used for biosensing. The biosensing refers to a technique for detecting a predetermined substance in vivo. An example of biosensing can be a technique for quantitatively detecting a predetermined molecule by using the property of a biochemical molecule, such as an enzyme responsible for in-vivo chemical reaction and an antibody responsible for an immune reaction, to specifically react to the predetermined molecule. The substance used for the biosensing, more specifically, used in the system of detecting a predetermined substance to be detected, is called a biosensing element.

Generally, the biosensing element is constituted of a molecular recognition motif which binds to a substance to be detected and a signal transduction motif converting a binding signal into a detectable signal. Detection of a disease-related substance by a biosensing technique has been expected to be used in various cases, for example, early detection of a disease such as cancer and health check such as blood glucose measurement typically in diabetes patients.

Examples of the molecular recognition motif in existing biosensing elements include proteins such as antibodies and nucleic acid molecules such as nucleic acid aptamers. The nucleic acid aptamer refers to a nucleic acid molecule that highly specifically binds to various substances such as ions, low-molecular compounds, and proteins. The nucleic acid molecule has the property to bind to a complementary nucleotide sequence. Due to this property, the nucleic acid molecule can be used as the molecular recognition motif when a target nucleic acid having a predetermined nucleotide sequence is used as a substance to be detected.

In the meantime, a complex of a nucleic acid and a protein can be immobilized onto a substrate by introducing a thiol group into the terminal of the nucleic acid molecule. In other words, when a complex of a nucleic acid and a protein is used, the protein can be immobilized onto a substrate via the nucleic acid molecule. Likewise, when proteins functioning as a signal transduction motif are immobilized onto a substrate, a device having a plurality of biosensing elements can be constructed.

### PRIOR ART

### Patent Literature

Patent Literature 1: Korean Patent No. 10-2020-0084690

### Non Patent Literature

Non Patent Literature 1: Sang, P. B.; Srinath, T.; Patil, A. G.; Woo, E. J.; Varshney, U. A unique uracil-DNA binding protein of the uracil DNA glycosylase superfamily. Nucleic acids research, 2015, 43 (17), 8452-8463.

### SUMMARY OF INVENTION

### Object to be Achieved

Patent Literature 1 discloses a complex of a nucleic acid and a protein, and the complex has a structure where UdgX having a nucleic acid molecule bound thereto and an antibody are connected via protein A. More specifically, a protein of interest is associated via a non-covalent bond, that is, not strongly bound. For this reason, the complex disclosed in Patent Literature 1 is impractical. In addition, the binding to an antibody is specified exclusively via protein A. For this reason, the complex lacks versatility.

In the circumstance mentioned above, an object of the present invention is to provide a protein-nucleic acid complex having a nucleic acid molecule and a protein covalently bound to each other, and not limited in the type of protein, a reagent and method for detecting a substance using the protein-nucleic acid complex, and a method for using the protein-nucleic acid complex.

### Means to Achieve Object

The present inventors conducted intensive studies with a view to achieving the object mentioned above. As a result, they found that when uracil DNA glycosylase covalently bound to a nucleic acid molecule is covalently bound directly or indirectly to a protein of interest, a protein-nucleic acid complex having the protein and the nucleic acid molecule covalently bound to each other can be obtained regardless of the type of a protein of interest. Based on the finding, the present invention was accomplished.

The present invention comprises the followings.

(1) A protein-nucleic acid complex comprising a nucleic acid molecule having uracil, a uracil DNA glycosylase domain covalently bound to the nucleic acid molecule, and a protein domain covalently bound directly or indirectly to the uracil DNA glycosylase domain.

(2) The protein-nucleic acid complex according to (1), wherein the uracil DNA glycosylase domain and the protein domain are covalently bound via a tag peptide and a tag capture peptide.

(3) The protein-nucleic acid complex according to (1), wherein the nucleic acid molecule is a nucleic acid aptamer molecule which specifically binds to a predetermined substance or a molecule comprising a nucleotide sequence complementary to a predetermined nucleotide sequence.

(4) The protein-nucleic acid complex according to (1), wherein the nucleic acid molecule is a chemically modified nucleic acid molecule.

(5) A biosensing element comprising the protein-nucleic acid complex according to any one of (1) to (4).

(6) The biosensing element according to (5), wherein the biosensing element detects a substance to which the nucleic acid molecule comprised in the protein-nucleic acid complex specifically binds.

(7) The biosensing element according to (5), wherein the biosensing element detects a substance to which the protein domain comprised in the protein-nucleic acid complex specifically binds.

(8) The biosensing element according to (5), wherein the nucleic acid molecule comprised in the protein-nucleic acid complex is an aptamer molecule which increases peroxidase activity of a heme protein, and the protein domain comprised in the above protein-nucleic acid complex is an enzyme having oxidase activity which catalyzes an oxidation reaction to generate hydrogen peroxide.

(9) The biosensing element according to (5), wherein the protein-nucleic acid complex is immobilized onto a substrate via chemical modification of the nucleic acid molecule comprised in the protein-nucleic acid complex.

(10) A kit for detecting a substance, comprising
a protein-nucleic acid complex according to any one of the above (1) to (4), in which a protein domain is an enzyme having oxidase activity which catalyzes an oxidation reaction to generate hydrogen peroxide,
a first substrate as a substrate for the first enzyme, and
a second substrate as a substrate for a substrate to be detected to which the nucleic acid molecule of the protein-nucleic acid complex binds and which has peroxidase activity.

(11) The detection kit according to (10), wherein the enzyme and the first substrate are glucose oxidase and glucose, respectively; and the substance to be detected and the second substrate are hemoglobin and luminol, respectively.

(12) The detection kit according to (10), wherein the enzyme is a fusion protein with an antibody to the substance to be detected.

(13) A method for detecting a substance, comprising the steps of:
mixing the detection kit according to any one of (10) to (12) and a sample that possibly comprises a substance to be detected; and
measuring an enzyme reaction consuming the second substrate comprised in the detection kit,
wherein the substance to be detected in the sample is detected based on the enzyme reaction.

The specification includes the contents disclosed in JP Patent Application No. 2022-138531 based on which the priority of the present application claims.

### Advantageous Effects of Invention

According to the present invention, it is possible to provide a protein-nucleic acid complex having a nucleic acid molecule and a protein domain covalently bound to each other. The protein-nucleic acid complex according to the present invention is not limited by the type of protein and any type of protein can be used as the protein domain. Accordingly, the protein-nucleic acid complex according to the present invention prepared by using a protein-nucleic acid complex excellent in practicality and versatility, can be used for providing a kit and a method for detecting a substance excellent in detection sensitivity.

### BRIEF DESCRIPTION OF DRAWINGS

FIG. 1 is a diagram schematically showing a protein-nucleic acid complex to which the present invention is applied.

FIG. 2 is a diagram schematically showing a system for detecting hemoglobin by a biosensing element using a protein-nucleic acid complex to which the present invention is applied.

FIG. 3 is a diagram schematically showing a system for detecting hemoglobin by another biosensing element using a protein-nucleic acid complex to which the present invention is applied.

FIG. 4 is a diagram schematically showing a system for detecting methylation of genomic DNA by a biosensing element using a protein-nucleic acid complex to which the present invention is applied.

FIG. 5 is a diagram schematically showing another biosensing element using a protein-nucleic acid complex to which the present invention is applied.

FIG. 6 shows diagrams schematically showing vectors expressing various DNA-covalently binding proteins constructed in Examples.

FIG. 7 is a characteristic graph showing the calculation results of DNA binding efficiency of various DNA-covalently binding proteins synthesized in Examples.

FIG. 8 is a characteristic graph showing the nucleotide sequence encoding UdgX-SpyTag prepared in Examples and the amino acid sequence thereof.

FIG. 9 is a characteristic graph showing the nucleotide sequence encoding UdgX-SpyCatcher prepared in Examples and the amino acid sequence thereof.

FIG. 10 is a characteristic graph showing the evaluation results on the DNA binding ability of aptamer-GOx complexes prepared in Examples.

FIG. 11 is a characteristic graph showing the evaluation results on the oxidase activity of the aptamer-GOx complexes prepared in Examples.

FIG. 12 is a characteristic graph showing the evaluation results on the Hb detection sensitivity of the aptamer-GOx complexes prepared in Examples, when a low protein adsorption plate made of polypropylene or polystyrene was used.

FIG. 13 is a characteristic graph showing the detection results of Hb comprised in the sera derived from humans by the aptamer-GOx complexes prepared in Examples.

FIG. 14 is an electrophoretic photograph showing the SDS-PAGE analysis results when a SpyTag/SpyCatcher reaction was performed using UdgX-SpyTag and GDH-SpyCatcher.

FIG. 15 is an electrophoretic photograph showing the SDS-PAGE analysis results and fluorescence detection results when a UdgX-GDH complex and a TBA15 aptamer were reacted.

FIG. 16 is an electrophoretic photograph showing the SDS-PAGE analysis results when a SpyTag/SpyCatcher reaction was performed using UdgX-SpyTag and GOx-SpyCatcher.

FIG. 17 is an electrophoretic photograph showing the SDS-PAGE analysis results and fluorescence detection results when a UdgX-GOx complex and a TBA15 aptamer were reacted.

FIG. 18 is an electrophoretic photograph showing the SDS-PAGE analysis results when a SpyTag/SpyCatcher reaction was performed using UdgX-SpyTag and GDH-SpyCatcher.

FIG. 19 is an electrophoretic photograph showing the SDS-PAGE analysis results and fluorescence detection results when a UdgX-GDH complex and a PEA3-01 aptamer were reacted.

### DESCRIPTION OF EMBODIMENTS

The present invention will be described in followings in detail with reference to the Figures.

### [Protein-Nucleic Acid Complex]

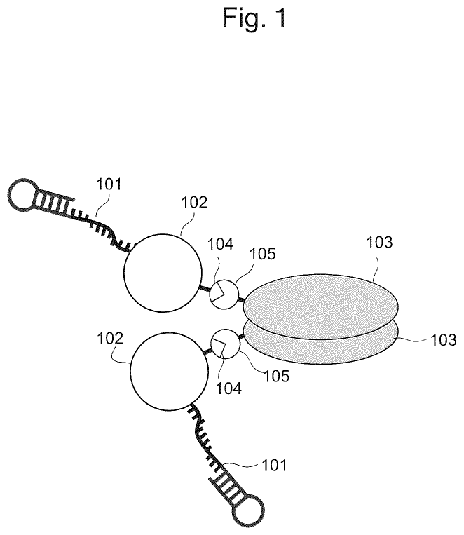

The protein-nucleic acid complex according to the present invention, as schematically shown in FIG. 1, has a nucleic acid molecule 101 having uracil, a uracil DNA glycosylase domain 102 covalently bound to the nucleic acid molecule 101, and a protein domain 103 covalently bound directly or indirectly to the uracil DNA glycosylase domain 102. Note that, FIG. 1 illustrates a structure when the protein domain 103 is a dimer, exemplarily. The protein-nucleic acid complex according to the present invention is not limited to the case where the protein domain 103 is a dimer and may be present in the form of a monomer or a multimer such as a trimer or a tetramer.

In the case shown in FIG. 1, the uracil DNA glycosylase domain 102 and the protein domain 103 are covalently bound indirectly. The case of “covalently bound indirectly” includes a case where proteins are covalently bound via a peptide. For example, using a so-called tag peptide 104 and a tag catcher peptide 105 (also referred to as Tag peptide and Catcher peptide), the uracil DNA glycosylase domain 102 and the protein domain 103 can be covalently bound. More specifically, either one of the tag peptide 104 and the tag capture peptide 105 is introduced into the C terminal or the N terminal of the uracil DNA glycosylase domain 102, whereas the other one is introduced into the C terminal or the N terminal of the protein domain 103. Then, the uracil DNA glycosylase domain 102 having either one of the tag peptide 104 and the tag capture peptide 105 and the protein domain 103 having the other one are mixed to form a covalent bond between the tag peptide 104 and the tag capture peptide 105.

Examples of the tag peptide 104 and the tag capture peptide 105 may include SpyTag/SpyCatcher system (Bijan Zakeri et al., PNAS, 109 (12) E690-E697, 2012) and SnoopCatcher/SnoopTag system (Gianluca Veggiani et al., PNAS, 113 (5) 1202-1207, 2016). As the tag peptide and the tag capture peptide, an improved the above SpyTag/SpyCatcher system, such as SpyTag2/SpyCatcher2 system (Keeble et al., Angew. Chem. Int. Ed. 2017, 56, 16521-16525) and SpyTag3/SpyCatcher3 system (Keeble et al., PNAS, 116 (52) 26523-26533, 2019) can be used.

Different from the case shown in FIG. 1, the uracil DNA glycosylase domain 102 and the protein domain 103 may be covalently bound directly. The case where “the uracil DNA glycosylase domain 102 and the protein domain 103 are covalently bound directly” includes a case where a fusion protein of the uracil DNA glycosylase domain 102 and the protein domain 103 is synthesized. In the case where a fusion protein of the uracil DNA glycosylase domain 102 and the protein domain 103 is synthesized, a linker sequence (e.g., GSG linker sequence) may be arranged between the uracil DNA glycosylase domain 102 and the protein domain 103 or the uracil DNA glycosylase domain 102 and the protein domain 103 may be connected without a linker sequence between them.

### [Nucleic Acid Molecule]

The nucleic acid molecule 101 has a uracil residue in the molecule for covalently binding to the uracil DNA glycosylase domain 102. The nucleotide sequence of the nucleic acid molecule 101 is not particularly limited, and any nucleotide sequence may be used. The nucleic acid molecule 101 may be a molecule comprising a nucleic acid aptamer molecule which specifically binds to a predetermined substance or a molecule comprising a complementary nucleotide sequence to a predetermined nucleotide sequence. When the nucleic acid molecule 101 is a molecule comprising a nucleic acid aptamer molecule or a complementary nucleotide sequence to a predetermined nucleotide sequence, the nucleic acid molecule 101 can be easily prepared by chemical synthesis as long as the nucleotide sequence thereof is determined.

The nucleic acid aptamer molecule refers to a molecule having a guanine quadruplex structure and capable of binding to a predetermined target substance. Examples of the target substance can include, but are not particularly limited to, proteins such as a growth factor, an enzyme, a receptor, a membrane protein, and a virus protein. Examples of the target substance can include cells presenting a protein on the surface such as microorganisms, cancer cells, iPS cells, and ES cells. As an example, the nucleic acid aptamer molecule can be a nucleic acid molecule consisting of a polynucleotide having a predetermined nucleotide sequence and having a function to enhance the peroxidase activity of a heme protein. The nucleic acid molecule 101 may be constituted of two linked nucleic acid aptamer molecules which bind to different target substances.

The nucleic acid molecule 101 may have one or a plurality of nucleotides added to the 5′ end and/or 3′ end of a nucleic acid aptamer molecule. Generally, the reason why an aptamer specifically binds to a target substance is considered that the aptamer forms a predetermined structure (three-dimensional structure or planar structure). Accordingly, the binding activity to a target substance can be maintained even if a nucleotide sequence not forming the predetermined structure is added to one or both ends of the nucleotide sequence of the nucleic acid aptamer molecule. For example, 1 to 3 natural bases except ACGT, such as inosine or a methylated cytosine or non-natural bases highly hydrophobic can be comprised at one or both ends of the nucleic acid aptamer molecule. Whether a specific structure is presented by a predetermined nucleotide sequence can be analyzed by computer software RAMAPO COLLEGE Mapper (Nucleic Acids Research 2006 July; 34 (Web Server issue): W676-W682) (for example, open to public at http://bioinformatics.ramapo.edu/QGRS/analyze.php). For example, poly t sequence is widely known as the nucleotide sequence not forming a predetermined structure. Accordingly, nucleic acid aptamer molecule having a poly t sequence added to one or both ends can maintain a specific binding activity to a target substance.

Examples of the nucleic acid aptamer molecule to be used as the nucleic acid molecule 101 include an aptamer molecule disclosed in JP Patent Publication (Kokai) No. 2017-200472A. The nucleic acid aptamer molecule is a nucleic acid molecule having a predetermined nucleotide sequence and consisting of a polynucleotide having a function to enhance the peroxidase activity of a heme protein. As shown in JP Patent Publication (Kokai) No. 2017-200472A, the nucleic acid aptamer molecule having a function to enhance the peroxidase activity of a heme protein has a guanine quadruplex structure (also referred to as a G4 structure). Generally, it is known that the aptamer having a G4 structure (also referred to as a G4 aptamer) includes an aptamer of parallel type G4 and an aptamer of anti-parallel type G4. As a result of circular dichroism (CD) spectral measurement, it has been confirmed that the nucleic acid aptamer molecule herein is a parallel type G4, as disclosed in JP Patent Publication (Kokai) No. 2017-200472A. Note that, in the G4 aptamer, 4 guanines are arranged in parallel on a plane to form G-quartet and a target substance binds so as to intrude into the G-quartets. Examples of the heme protein on which the nucleic acid aptamer molecule acts include myoglobin, hemoglobin, metmyoglobin, catalase, and cytochrome p450. Of them, myoglobin and hemoglobin are preferable.

As the nucleic acid aptamer molecule to be used as the nucleic acid molecule 101, for example, a nucleic acid aptamer molecule having a binding ability to a receptor binding domain (RBD) of a viral spike protein can be used. The spike protein refers to a glycoprotein. A plurality of spike proteins are almost uniformly present in the viral surface and interact with the cell surface receptor of a host cell. Note that, the receptor binding domain (RBD) refers to a region interacting with the cell surface receptor of a host cell. Examples of the virus having a spike protein can include viruses represented by a seasonal influenza virus, a new influenza virus, a coronavirus, a new coronavirus, an orthomyxovirus, a paramyxovirus, a rhabdovirus, a filovirus, a bunyavirus, an arenavirus, and a retrovirus.

The nucleic acid molecule 101 may have a chemical modification at an end or in the middle of the molecular chain. Examples of the chemical modifications (will be described later in detail) can include introduction of a thiol group for immobilization to a substrate into an end and biotinylation of an end. Furthermore, the nucleic acid molecule 101 can be chemically modified by, e.g., a fluorescence substance or a chemical agent.

Since the nucleic acid molecule according to the present invention is DNA having a uracil residue, it can be easily prepared by chemical synthesis using, e.g., a commercially available DNA synthesis device.

### [Protein Domain]

The protein domain 103 is not particularly limited and may be a protein having any amino acid sequence. The protein constituting the protein domain 103 may have a specific affinity to a predetermined substance, an enzyme having a predetermined enzyme activity or a fusion protein of a protein having a specific affinity to a predetermined substance and an enzyme having a predetermined enzyme activity. Examples of the protein having specific affinity to a predetermined substance can include, but are not particularly limited to, typically antibodies. The antibody may be an antibody to a target substance to which the nucleic acid molecule 101 binds, or an antibody to a growth factor, an enzyme, a receptor, or a membrane protein. Also, the antibody may be an anti-methylated DNA antibody or a so-called molecular target therapeutic agent for cancer treatment, such as an anti-EGFR antibody, an anti-HER2 antibody, or an anti-PD-1 antibody.

When an enzyme having a predetermined enzyme activity is used as the protein domain 103, the enzyme is not particularly limited but an enzyme catalyzing an enzyme reaction using a predetermined substance as a substrate (first substrate) to generate hydrogen peroxide, that is, an enzyme having oxidase activity can be mentioned. Examples of the enzyme having oxidase activity that can be used herein include glucose oxidase, aldehyde oxidase, cytochrome oxidase, cytochrome oxidase, catechol oxidase, diphenol oxidase, cholesterol oxidase, cytochrome C oxidase, hypoxanthine oxidase, xanthine oxidase, NADPH oxidase, lactate oxidase, galactose oxidase, and alcohol oxidase. Of them, lactate oxidase or glucose oxidase is preferably used as the enzyme having oxidase activity.

As the protein domain 103, other than the protein having a specific affinity to a predetermined substance and the enzyme having a predetermined enzyme activity, a protein generating a signal, such as a fluorescent protein, can be used. Examples of the fluorescent protein can include luciferase and a circularly-permuted fluorescent protein mutant such as cp luciferase.

### [Biosensing Element]

The protein-nucleic acid complex mentioned above can be used as a biosensing element having a function to sense a substance. The biosensing element is generally constituted of a molecular recognition motif which binds to a substance to be detected and a signal transduction motif converting a binding signal into a detectable signal. In the case where the protein-nucleic acid complex mentioned above is used as a biosensing element, either one of the nucleic acid molecule 101 and the protein domain 103 is used as the molecular recognition motif and the other is used as the signal transduction motif. In other words, the protein-nucleic acid complex may be used as a biosensing element for detecting a target substance to which the nucleic acid molecule 101 binds or for detecting a substance to which the protein domain 103 binds.

In the case of a biosensing element for detecting a target substance to which the nucleic acid molecule 101 binds, if a fluorescent protein or an enzyme generating a detectable substance is used as the protein domain 103, the protein domain 103 can be used as a signal transduction motif. Conversely, in the case of a biosensing element for detecting a target substance to which the protein domain 103 binds, if an aptamer molecule which binds to a fluorescently labeled ligand is used as the nucleic acid molecule 101, the nucleic acid molecule 101 can be used as a signal transduction motif.

The protein-nucleic acid complex mentioned above, if a molecule having a thiol group at an end is used as the nucleic acid molecule 101, can be immobilized onto a substrate via the thiol group. Note that, if the end of the nucleic acid molecule 101 is biotinylated, the nucleic acid molecule 101 can be immobilized onto a substrate having avidin. At this time, the protein domain 103 in the protein-nucleic acid complex plays a role as a molecular recognition motif and a signal transduction motif, and the nucleic acid molecule 101 contributes only to the immobilization onto a substrate.

### Example 1 of Biosensing Element

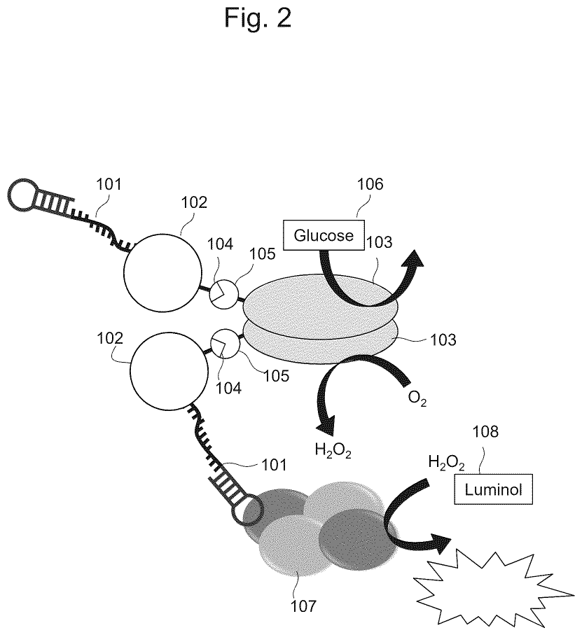

An example of the biosensing element using the protein-nucleic acid complex mentioned above, a biosensing element highly precisely detecting hemoglobin will be described. The system of the element is schematically shown in FIG. 2. This system, as shown in FIG. 2, includes the protein-nucleic acid complex shown in FIG. 1, a substrate 106 (first substrate 106) of the protein domain 103, that is, an enzyme having oxidase activity, and a second substrate 108 serving as a substrate for peroxidase in a detection-target hemoglobin 107. The protein domain 103 herein has oxidase activity and catalyzes an enzyme reaction of a predetermined substance used as a substrate (first substrate 106) to generate hydrogen peroxide.

The first substrate 106 can be appropriately selected for use depending on the type of protein domain 103. A pair of the protein domain 103 and the first substrate 106 is preferably a pair of glucose oxidase and glucose or a pair of lactate oxidase and lactic acid. This is because glucose and lactic acid as the first substrate 106 are stable compounds and inexpensive.

As shown in FIG. 2, this system includes the second substrate 108 serving as a substrate of hemoglobin 107 having peroxidase activity. The second substrate 108 is preferably a substance that can optically detect particularly the progression of peroxidase reaction of hemoglobin 107. More specifically, as the second substrate 108, orthophenylenediamine or luminol can be used.

In this system constituted as mentioned above, that is, a reaction system in which a protein-nucleic acid complex (as shown in FIG. 1) comprising the nucleic acid molecule 101 capable of binding to hemoglobin 107 and the protein domain 103 having oxidase activity, the first substrate 106 and the second substrate 108 are present, a target substance, i.e., hemoglobin 107, can be detected. More specifically, if hemoglobin 107 is present in the reaction system, the nucleic acid molecule 101 of the protein-nucleic acid complex binds to hemoglobin 107 and the protein domain 103 of the protein-nucleic acid complex comes closer to hemoglobin 107. In this state, an oxidase reaction using the first substrate 106 is conducted by the protein domain 103 to generate hydrogen peroxide. Since hemoglobin 107 is present in the proximity of the protein domain 103, it can consume hydrogen peroxide generated from the enzyme reaction by the protein domain 103. In this manner, the peroxidase reaction using the second substrate 108 by hemoglobin 107 proceeds.

Accordingly, according to this system, hemoglobin 107 can be detected by measuring the peroxidase reaction using the second substrate 108 by hemoglobin 107. For example, in the case where an optically detectable substance such as luminol is used as the second substrate 108, hemoglobin 107 can be detected by measuring chemiluminescence generated by a so-called luminol reaction. Note that, the means for measuring chemiluminescence is not particularly limited and chemiluminescence may be measured visually or by a method for measuring ultra-weak chemiluminescence.

In contrast, if hemoglobin 107 is not present in the reaction system in which the protein-nucleic acid complex comprising the nucleic acid molecule 101 capable of binding to hemoglobin 107, and the protein domain 103 having oxidase activity, the first substrate 106 and the second substrate 108 are present, an oxidase reaction using the first substrate 106 by the protein domain 103 proceeds to generate hydrogen peroxide but the peroxidase reaction using the second substrate 108 by hemoglobin 107 will not proceed.

In the detection method for hemoglobin 107 using this system mentioned above, at least, a first reagent comprising the first substrate 106 and a second reagent comprising the protein-nucleic acid complex can be used. Note that, components (for example, the second substrate 108) other than the first substrate 106 and the protein-nucleic acid complex may be comprised in either one or both of the first reagent and second reagent. Since the first reagent and the second reagent are separately comprised, progression of the oxidase reaction using the first substrate 106 by the protein domain 103 in the protein-nucleic acid complex can be suppressed during storage. When the first reagent and the second reagent are mixed, the oxidase reaction using the first substrate 106 by the protein domain 103 in the protein-nucleic acid complex can proceed to detect hemoglobin 107 as mentioned above.

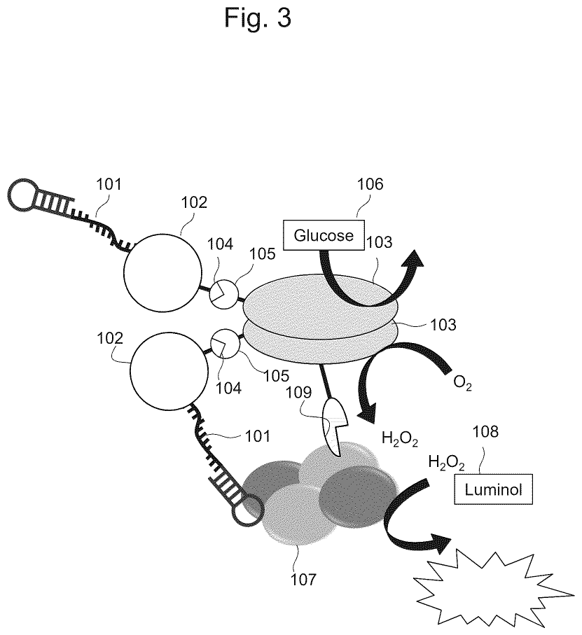

The constitution of the biosensing element is not limited to that shown in FIG. 2. As shown in FIG. 3, the protein domain 103 may be a fusion protein of an antibody 109 to a detection-target hemoglobin 107, and an enzyme having oxidase activity. In this case, since the antibody 109 and the nucleic acid molecule 101 differ in interface for recognizing hemoglobin 107, if these are cooperatively used, stronger binding that cannot be achieved by a combination of antibodies can be attained, with the result that hemoglobin 107 can be detected with higher sensitivity.

### Example 2 of Biosensing Element

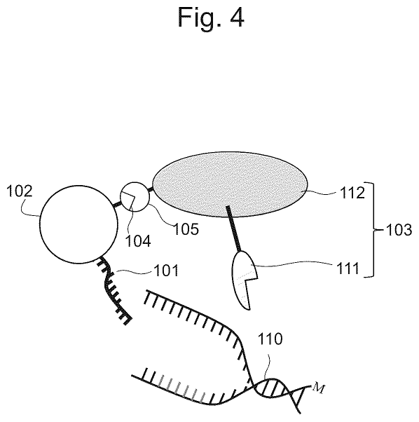

Another example of the biosensing element using the protein-nucleic acid complex mentioned above, a biosensing element for highly precisely detecting methylation at a predetermined position of genomic DNA will be described. This system is schematically shown in FIG. 4. The protein-nucleic acid complex used in this system has the nucleic acid molecule 101 having a nucleotide sequence that hybridizes with a site in the proximity of a position (in FIG. 4, M) at which methylation of genomic DNA 110 is to be detected, and the protein domain 103 consisting of a fusion protein of an antibody 111 to anti-methylated DNA and a fluorescent protein 112. As the fluorescent protein 112 herein, luciferase (cp luciferase) lost in function by circular permutation can be used.

According to the system shown in FIG. 4, the nucleic acid molecule 101 hybridizes with a predetermined region of the genomic DNA 110; at the same time, if methylation is present at a predetermined position, an antibody 111 to the anti-methylated DNA binds to methylated DNA. When the binding is formed, the enzyme activity of cp luciferase is recovered and luciferase luminescence can be observed. Accordingly, when the protein-nucleic acid complex constituted as mentioned above is used, methylation of a predetermined position in the genomic DNA 110 can be highly sensitively detected.

### Example 3 of Biosensing Element

Another example of the biosensing element using the protein-nucleic acid complex mentioned above can be a biosensing element for highly precisely detecting EGFR, HER2 or PD-1 highly expressed in cancer. In this case, the protein-nucleic acid complex has the protein domain 103 comprising an anti-EGFR antibody, an anti-HER2 antibody or an anti-PD-1 antibody, the nucleic acid molecule 101 modified with a labeled probe or the nucleic acid molecule 101 bound to a fluorescent protein. Using such a protein-nucleic acid complex, cancer cells overexpressing EGFR, HER2 or PD-1 can be highly precisely detected.

In this case, the protein domain 103 is preferably a fusion protein of an antibody such as an anti-EGFR antibody, an anti-HER2 antibody or an anti-PD-1 antibody and lactate oxidase. When the protein domain 103 exhibits lactate oxidase activity, lactic acid around cancer cells can be consumed, thereby inhibiting angiogenesis and proliferation of cancer cells. Simultaneously, hydrogen peroxide generated by lactate oxidase can give damage to the cancer cells, promoting death of cancer cells. Use of the protein-nucleic acid complex enables not only detecting cancer but also treating cancer. The protein-nucleic acid complex can be used as a theranostics molecule.

In particular, in the case where the protein domain 103 is a fusion protein of the above antibody and lactate oxidase, since the lactate oxidase forms a multimer (for example, tetramer), the antibody can serve as a multivalent antibody, with the result that dense accumulation can be attained and high cancer-growth suppression effect can be expected.

### Example 4 of Biosensing Element

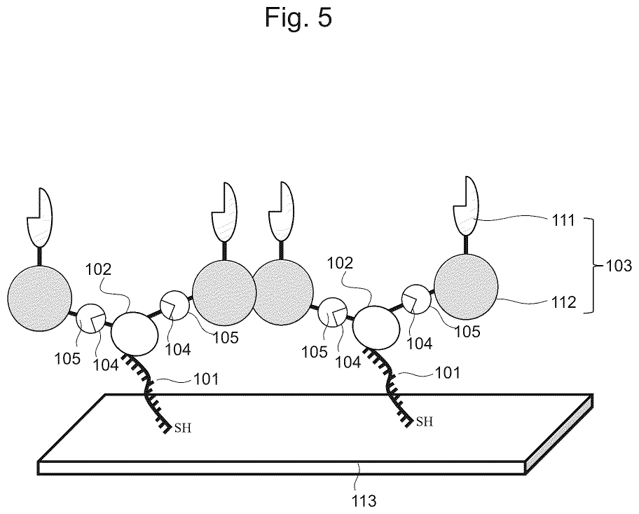

Another example of the biosensing element using the protein-nucleic acid complex mentioned above is an element using a protein-nucleic acid complex having the nucleic acid molecule 101 with a thiol group at an end and a pair of the protein domains 103 indirectly connected via the uracil DNA glycosylase domain 102, the tag peptide 104 and the tag capture peptide 105, as shown in FIG. 5. More specifically, the protein-nucleic acid complex of this example has two protein domains 103 in a single molecule. The protein-nucleic acid complex thus constituted can be immobilized onto a substrate 113 via the thiol group. In this case, since the protein-nucleic acid complex has two protein domains 103 in a molecule, the complex can form a dimer or a multimer larger than a dimer together with the adjacent protein domain 103. Because of this, in the biosensing element using the protein-nucleic acid complex constructed in the way, the protein domain 103 are densely accumulated on the substrate 113.

In such a biosensing element, a detection-target substance can be detected by measuring a current value that is changed when the detection-target substance binds to the protein domain 103. More specifically, when the detection-target substance is a virus, the protein domain 103 may be a fusion protein of an antibody 114, which binds to a surface protein of the virus used as an antigen, and lactate oxidase 115. With this structure, in the state where the virus and the antibody 114 are bound, lactate oxidase 115 generates hydrogen peroxide with lactic acid serving as a biomarker for viral infection used as a substrate. When a current is measured at this time, the virus comprised in a specimen can be detected.

In particular, when the protein domain 103 is a fusion protein of the above antibody and lactate oxidase, since lactate oxidase forms a multimer (for example, tetramer), the antibody can serve as a multivalent antibody. As a result, sensitivity to a detection-target substance can be improved.

### Examples

The present invention will be more specifically described with reference to Examples, but the technical scope of the present invention is not limited to the following Examples.

### [Example 1: Evaluation of DNA-Binding Protein]

In this Example, to prepare a stoichiometric and stable protein-nucleic acid complex, attention was focused on DNA-covalently binding proteins. DNA-covalently binding proteins suitable for a protein-nucleic acid complex were tried to be found by searching a plurality of DNA-covalently binding proteins and evaluating its properties. In this Example, four DNA-covalently binding proteins different in binding mode, host and molecular weight, TYLCV, RepB, Tral and UdgX, were selected and subjected to evaluation of properties.

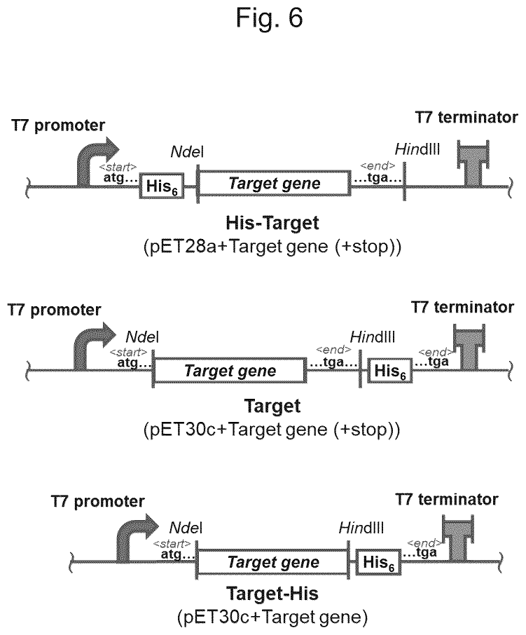

First, for the proteins TYLCV, RepB, and UdgX, genes synthesizing them were obtained based on literature information, whereas, for the protein Tral, F plasmid was extracted from E. coli to obtain a vector having a gene of interest. To two types of vectors (pET28a and pET30c) treated with restriction enzymes, NdeI and HindIII, two types of genes of interest (having and not having a stop codon at the C terminal) treated with the same restriction enzymes, were ligated. As a result, vectors expressing a DNA-covalently binding protein having His-tag attached to the N terminal, a DNA-covalently binding protein having a His-tag at the C terminal, and a DNA-covalently binding protein having no His-tag respectively were constructed (FIG. 6).

Subsequently, E. coli LOBSTR was transformed by the vector constructed by a heat shock method, and thereafter, seeded in LB agar medium comprising kanamycin (final concentration: 50 μg/mL) and cultured at 37 °C overnight. After culture, a transformant was inoculated in LB medium (1.5 mL) comprising kanamycin (final concentration: 50 μg/mL) and an autoinduction agent. After culture at 28 °C and 140 rpm overnight and cells were collected. A soluble fraction and an insoluble fraction were subjected to SDS-PAGE analysis. As a result, in the cases of TYLCV and RepB having a His-tag added to the C terminal, no bands were observed near a theoretical molecular weight of a DNA-covalently binding protein. From this, it was considered suitable that a His-tag is added to the N terminal. In the case of Tral, even if a His-tag was added to the N terminal or the C terminal, a band was observed near the theoretical molecular weight in both cases. Since the active center of Tral is present at the N terminal, it was considered suitable that a His-tag is added to the C terminal. In contrast, in the case of UdgX, a case where a His-tag was added to the C terminal in a predetermined culture condition, expression level in the soluble fraction was high. From this, it was considered suitable that His-tag is added to the C terminal.

Subsequently, to evaluate the DNA binding ability of the DNA-covalently binding protein obtained, the DNA-covalently binding proteins (TraI: final concentration of 500 nM, TYLCV, RepB, UdgX: final concentration of 4000 nM), and single-stranded DNAs having recognition sequences of individual DNA-covalently binding proteins modified with FAM were mixed in a reaction Buffer (Tris having a final concentration of 50 mM, NaCl having a final concentration of 100 mM, pH 7.2). Immediately after a specimen was mixed and incubated at 37 °C for one hour, the mixture was added in SDS-PAGE Sample Buffer and heat treatment was performed at 95 °C for 10 minutes to terminate the reaction. The resultant samples were subjected to SDS-PAGE analysis. After the electrophoresis, detection of fluorescence of FAM and staining with Coomassie Brilliant Blue (CBB) were performed. Note that, the above operations were all carried out in a dark room.

As a result of the SDS-PAGE analysis, bands corresponding to proteins of interest were observed at sites near the theoretical molecular weight in all lanes where DNA-covalently binding proteins were added, and in the lane of a DNA-covalently binding protein to which ss DNA was added, another band (2nd band) was observed at a higher molecular-weight range (data not shown). As a result of fluorescence observation, fluorescence derived from FAM modifying ssDNA was detected from the band at the higher molecular-weight range. From this, it was suggested that the DNA-covalently binding protein and ssDNA are covalently bound.

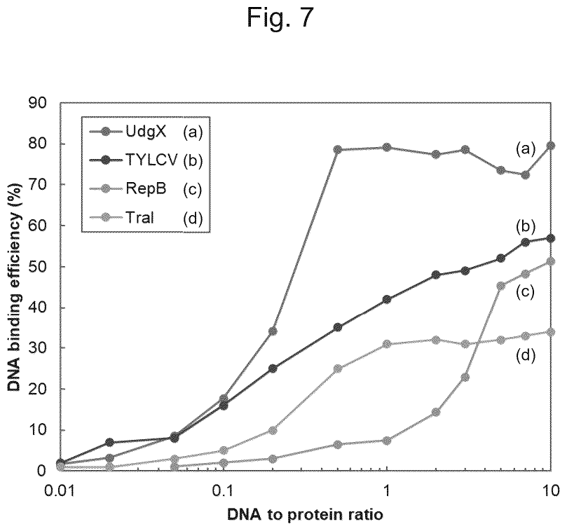

The same experiment was performed by setting the final concentrations of all DNA-covalently binding proteins equally to 4000 nM, and the ratio of the DNA-covalently binding protein and ssDNA was changed. The DNA binding efficiency was calculated and the results are shown in FIG. 7 and Table 1. Note that, the DNA binding efficiency was calculated by the following formula:

100×[intensity of DNA−covalently binding protein bound to ssDNA]/[intensity of DNA−covalently binding protein alone].

TABLE 1

| DNA- | | | DNA |
|---|---|---|---|
| covalently | | | binding |
| binding | | Productivity | efficiency |
| protein | Classification | [mg/L] | [%] |
| TYLCV | HUH endonuclease | 5.6 | 57.0 |
| RepB | | 50.1 | 51.3 |
| TraI | | 173.3 | 42.0 |
| UdgX | Uracil DNA | 16.8 | 93.2 |
| | glycosylase | | |

As shown in FIG. 7 and Table 1, it was confirmed that UdgX has particularly high DNA binding ability. This is considered because a reverse reaction intrinsic to HUH-endonuclease is not observed in uracil DNA glycosylase. Thus, in this Example, UdgX was successfully selected as the DNA-covalently binding protein suitable for the preparation of DNA-protein complex.

### [Example 2: Preparation/Evaluation of Protein-Nucleic Acid Complex]

In this Example, a DNA-protein complex was prepared using UdgX and tried to be applied to a sensing element. Human hemoglobin (Hb) was used as a detection target. A lower Hb concentration in the whole blood than a normal value indicates anemia, whereas a higher HB concentration indicates polycythemia. The Hb concentration is used as disease markers of, e.g., leukemia, heart disease and dehydration. Hb is also known to have peroxidase-like activity using hydrogen peroxide as a substrate and catalyzes the luminescence reaction of luminol. Note that, in this Example, an anti-Hb aptamer (PEA3-01) enhancing peroxidase activity disclosed in JP Patent Publication (Kokai) No. 2017-200472A was used.

Then, in this Example, a complex of an anti-Hb aptamer and glucose oxidase (GOx) using UdgX was prepared and tried to be used for detecting Hb. If a complex of an anti-Hb aptamer and GOx was formed, it was considered that when the anti-Hb aptamer binds to Hb, GOx and Hb come closer to each other, with the result that hydrogen peroxide generated by GOx is efficiently supplied to Hb (FIG. 2). The resulting chemiluminescence was expected to attain highly sensitive Hb detection. In preparation of the complex, UdgX and GOx were fused by use of a protein-protein connection module, SpyTag/SpyCatcher system. SpyTag and SpyCatcher can be just mixed to connect a protein to a protein by a covalent bond. Owing to this system, it was considered that an anti-Hb aptamer, UdgX and GOx can be covalently bound to form a complex and the complex can be universally applied as a stable sensing element.

### [Design of Nucleic Acid Molecule]

In this Example, as a nucleic acid molecule to be covalently bound to UdgX, a nucleic acid molecule was synthesized by adding, to an anti-Hb aptamer (PEA3-01), a UdgX recognition sequence having a uracil residue. The nucleic acid molecule synthesized has a nucleotide sequence: 5′-GGGCGGGTTGGGCTGGG-TTT-CTCAAGTGUAGGCATGCAAGAGCT-TTT-TCTACATCTCAGCATTCT-3′ (SEQ ID NO: 1). Note that the sequence from the 5′ end to the hyphen represents an anti-Hb aptamer (PEA3-01) and the sequence underlined represents a UdgX recognition sequence. UdgX binds to the 29th position, U (uracil) residue, from the 5′ end of this sequence.

### [Design of UdgX-SpyCatcher and UdgX-SpyTag]

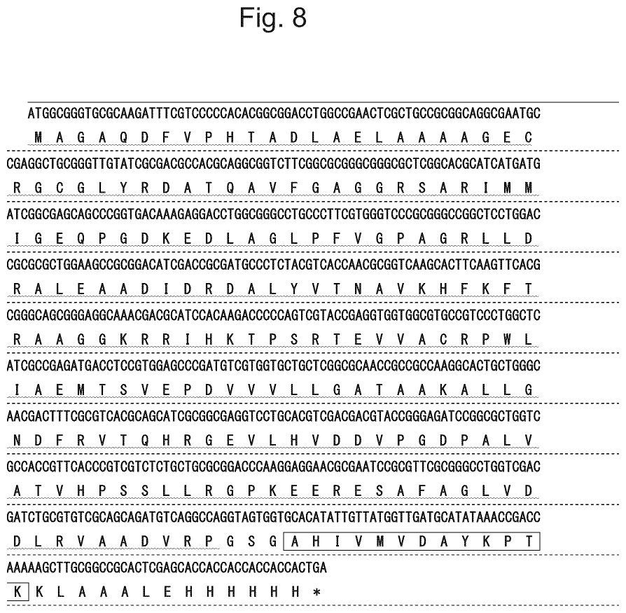

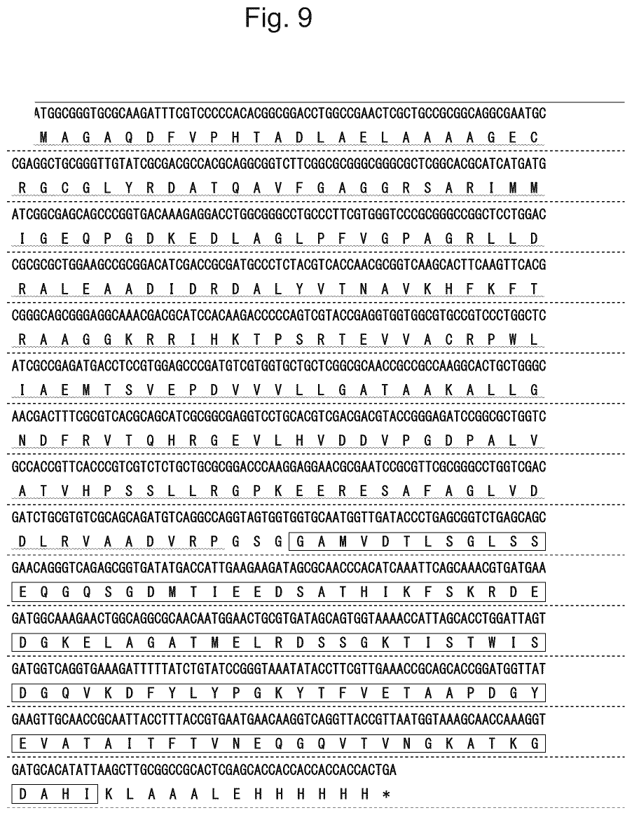

In this Example, it was found that DNA binding ability can be maintained high in the case where SpyCatcher or SpyTag was fused to the C terminal than the case where SpyCatcher or SpyTag was fused to the N terminal of UdgX (data not shown). For this reason, a protein was synthesized by fusing SpyCatcher or SpyTag to the C terminal of UdgX. The nucleotide sequence (SEQ ID NO: 2) encoding a fusion protein (UdgX-SpyTag) prepared by fusing SpyTag to the C terminal of UdgX and the amino acid sequence (SEQ ID NO: 3) of the fusion protein are shown in FIG. 8. The nucleotide sequence (SEQ ID NO: 4) encoding a fusion protein (UdgX-SpyCatcher) prepared by fusing SpyCatcher to the C terminal of UdgX and the amino acid sequence (SEQ ID NO: 5) of the fusion protein are shown in FIG. 9. Note that, in FIGS. 8 and 9, the amino acid sequence of UdgX is underlined by a wavy line and the amino acid sequences of SpyCatcher and SpyTag are framed.

### [Synthesis of UdgX-SpyCatcher and UdgX-SpyTag]

E. coli LOBSTR was transformed by vectors expressing UdgX-SpyTag and UdgX-SpyCatcher, seeded in LB agar medium comprising kanamycin (final concentration: 50 μg/mL), and cultured at 37 °C overnight. Each of the obtained transformants was inoculated in LB medium (100 mL) comprising kanamycin (final concentration: 50 μg/mL) and an Auto induction reagent. Note that, FeCl₃ (final concentration: 0.01%) and citric acid (mole ratio of FeCl₃ to citric acid was 1:2) were also added to the LB medium. Culture was performed at 28 °C and 140 rpm overnight and cells were collected. A soluble fraction and an insoluble fraction were subjected to SDS-PAGE analysis. As a result, highly purified UdgX-SpyTag and UdgX-SpyCatcher were obtained from the soluble fraction. The yields of UdgX-SpyTag and UdgX-SpyCatcher were calculated as 28.2 mg protein/L culture and 52.5 mg protein/L culture, respectively.

### [Evaluation on DNA Binding Ability of UdgX-SpyCatcher and UdgX-SpyTag]

The UdgX-SpyTag and UdgX-SpyCatcher obtained, and the recognition sequence (5′-CTCAAGTGUAGGCATGCAAGAGCT-3′, the 9th position from the 5′ end is uracil residue (SEQ ID NO: 6)) of UdgX were mixed in a reaction buffer (Tris having a final concentration of 50 mM, 100 mM NaCl, pH 7.2), incubated at 37 °C for one hour in a standing-still condition. Mixing with SDS-PAGE Sample Buffer followed by vortex was performed and heat treatment was performed at 95 °C for 10 minutes to terminate the reaction. The obtained sample was added to SDS-PAGE gel so as to comprise a total protein of 500 ng per lane and SDS-PAGE analysis was performed. Electrophoresis was performed at 20 mA for 90 minutes and fluorescence of FAM was detected. The resultant polyacrylamide gel was stained with Coomassie Brilliant Blue (CBB) and imaging was performed. In electrophoretic images obtained after CBB staining, quantification of bands was performed by ImageJ. Note that, the above operations were all carried out in a dark room. As a result, it was suggested that UdgX-SpyTag and UdgX-SpyCatcher obtained keep the same level of binding ability as UdgX fused only with a His-tag (data not shown).

### [Preparation of Protein-Nucleic Acid Complex]

A fusion protein (referred to as GOx-SpyCatcher) having glucose oxidase (glucose oxidase: GOx) and SpyCatcher in the order from the N terminal was prepared. Note that, the amino acid sequence of GOx-SpyCatcher is shown in SEQ ID NO: 7.

First, to prepare a UdgX-GOx complex, a SpyTag/SpyCatcher reaction was performed. Then, 5 μL of 4 μM UdgX-SpyTag and 5 μL of 2 μM GOx-SpyCatcher diluted with 20 mM Potassium phosphate buffers (P.P.B.) (pH 6.0) were mixed, and incubated (incubation time: 10 min, 30 min, 1 h, 2 h, and 24 h) at 4 °C at a standing-still condition. Herein, GOx-SpyCatcer was prepared by performing recombination production using E. coli, refolding from an insoluble fraction, followed by His-tag purification and then put in use. Finally, the obtained sample was subjected to SDS-PAGE analysis.

Subsequently, a protein-nucleic acid complex was prepared. The same amounts of 20 μM UdgX-SpyTag and 10 μM GOx-SpyCatcher diluted with 20 mM P.P.B. (pH 6.0) were mixed and incubated at 4 °C for 24 hours. To prepare an aptamer-GOx complex, a sequence having a UdgX recognition sequence, PEA3-01, and a fluorescent dye FAM, in combination was subjected to folding (95 °C for 10 min, 95 °C to 25 °C over 30 min) and then used for a DNA binding reaction. Thereafter, 5 μM UdgX-GOx complex and PEA3-01 having a 10 μM UdgX recognition sequence were mixed in a reaction buffer (10 mM Sodium phosphate buffer (pH 6.0) comprising 10 mM KCl) and incubated at 4 °C overnight. The obtained sample was subjected to SDS-PAGE analysis.

The electropherograms as the results of the SDS-PAGE analysis of these are not shown herein, but it was confirmed that there was a band near a theoretical molecular weight of a UdgX-GOx complex. From this, it was successfully confirmed that a UdgX-GOx complex was formed by a connection module, the SpyTag/SpyCatcher system. The binding reaction of the UdgX-GOx prepared and an aptamer was evaluated by SDS-PAGE analysis. As a result, a band was observed near a theoretical molecular weight of a complex formed by binding a nucleic acid molecule comprising PEA3-01 to the UdgX-Gox complex, although the electropherogram is also not shown. From this, it was suggested that a protein-nucleic acid complex (hereinafter referred to as aptamer-GOx complex) was successfully prepared.

### [Evaluation on DNA Binding Ability of Aptamer-GOx Complex]

First, 100 nM Hb was added to a 96-well plate at a rate 100 μL per well and incubated for one hour to immobilize Hb on the plate by adsorption. Washing was performed with Wash buffer (50 mM Tris, 138 mM NaCl, 2.7 mM KCl, 0.05% Tween 20, pH 8.0), and thereafter, 2% (w/v) Skimmed milk was added and incubation was performed for one hour at room temperature to block the plate. Washing was performed with Wash buffer and an aptamer-GOx complex was added at a rate of 100 μL per well and incubated for one hour at room temperature. Washing was performed three times with Wash buffer and the fluorescent of FAM derived from a FAM-modified aptamer was measured by a plate reader.

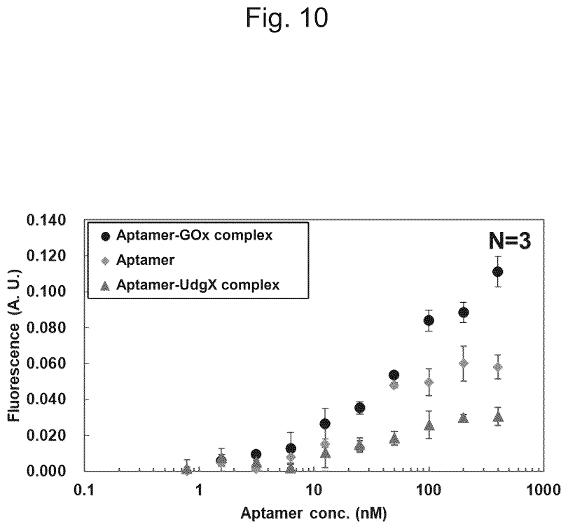

The measurement results of the fluorescence is shown in FIG. 10. As shown in FIG. 10, an increase in fluorescence intensity was observed in a concentration dependent manner of PEA3-01 to which a UdgX-GOx complex was bound. From this, it was suggested that PEA3-01 to which UdgX-GOx was bound has a binding ability to Hb. Furthermore, a Kd value was calculated. As a result, the Kd value of the case having an aptamer alone was 38 nM; that of the aptamer UdgX complex was 45 nM, and that of aptamer-GOx complex was 34 nM. The reason why the aptamer-GOx complex was slightly improved in binding ability was considered as follows. Since GOx forms a dimer, two aptamers which binds to Hb are present in a single molecule, so that dissociation rate decreased, compared to the case where a single binding site is present per molecule.

### [Evaluation on Enzyme Activity of Aptamer-GOx Complex]

After the aptamer-GOx complex was mixed with a reaction solution (HRP having a final concentration of 2 U/mL, TOOS having a final concentration of 1.5 mM, 4AA having a final concentration of 1.5 mM, and P.P.B. (pH 7.0) having a final concentration of 10 mM), glucose solutions different in concentration were reacted. In this manner, the oxidase activity of GOx was evaluated. Also the GOx-SpyCatcher and the UdgX-GOx complex were subjected to the same evaluation.

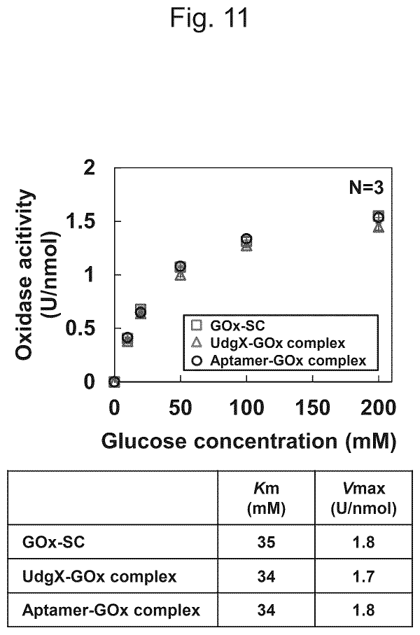

The evaluation results of the oxidase activity are shown in FIG. 11. As shown in FIG. 11, it was suggested that the activity of GOx in the aptamer-GOx complex is maintained compared to that of GOx having SpyCatcher fused to the C terminal.

### [Detection of Hb Using Aptamer-GOx Complex]

To study about a 96-well plate for use in Hb detection, Nunc™ 96-Well Polypropylene Storage Microplates (manufactured by ThermoFisher Scientific) and a low protein adsorption plate made of polystyrene, Low-binding surface (LBS) OptiPlate, and 96-well (white) (manufactured by PerkinElmer) were subjected to the following experiments.

An aptamer-GOx complex was similarly prepared using an aptamer having a UdgX recognition sequence not modified with FAM. Thereafter, the aptamer-GOx complex, which was diluted with a 10 mM Sodium phosphate buffer (pH 6.0) comprising 10 mM KCl so as to have a final concentration of 50 nM, and Hb, which was diluted with the same buffer so as to have a final concentration of 12.5 nM, were mixed on a 96-well plate so as to obtain a total of 20 μL. The mixture was incubated for one hour at room temperature, and 80 μL of reaction solution (glucose having a final concentration of 100 mM, luminol having a final concentration of 100 μM, and catalase having a final concentration of 0.1 nM, pH 8.0) was added. The chemiluminescence generated was measured by a plate reader.

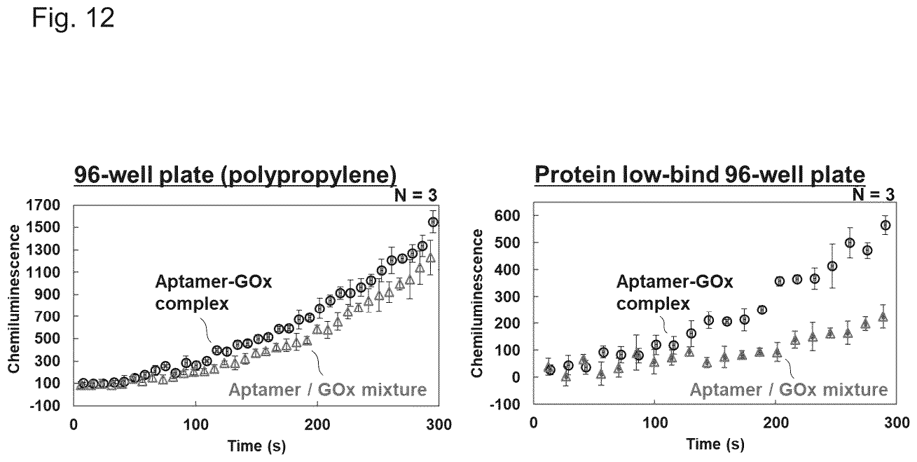

Hb-dependent chemiluminescence was observed by use of the low protein adsorption 96-well plate made of polystyrene. As a result, high-intensity chemiluminescence was observed in a case of an aptamer-GOx complex using UdgX, compared to a control in which an aptamer and GOx were mixed (FIG. 12, right figure). The difference in chemiluminescence intensity between both cases was larger than that in the case of using a 96-well plate made of polypropylene (FIG. 12, left figure). This was considered that chemiluminescence, which was generated by Hb and GOx adsorbed onto a plate and thereby closely positioned, is decreased by using a low protein adsorption 96-well plate, with the result that the effect produced by Hb and GOx closely positioned due to an aptamer was easily observed. From the results, it was suggested that when Hb is detected by an aptamer-GOx complex using a plate, a low protein adsorption plate made of polystyrene is preferably used.

### [Detection of Human-Serum Hb Using Aptamer-GOx Complex]

The following experiment was performed using a low protein adsorption plate made of polystyrene and Low-binding surface (LBS) OptiPlate, 96-well (white) (manufactured by PerkinElmer). An aptamer-GOx complex was similarly prepared using an aptamer having a UdgX recognition sequence not modified with FAM. The aptamer-GOx complex diluted with a 10 mM Sodium phosphate buffer (pH 6.0) comprising 10 mM KCl so as to have a final concentration of 50 nM and each of the Hb samples different in concentration and prepared by diluting Hb with human serum (1.0×10⁴ fold dilution) as a solvent were mixed on a 96-well plate so as to obtain a total of 20 μL and incubated for one hour at room temperature. A reaction solution (80 μL, glucose having a final concentration of 100 mM, and luminol having a final concentration of 100 μM, pH 8.0) was added and the chemiluminescence generated was measured by a plate reader.

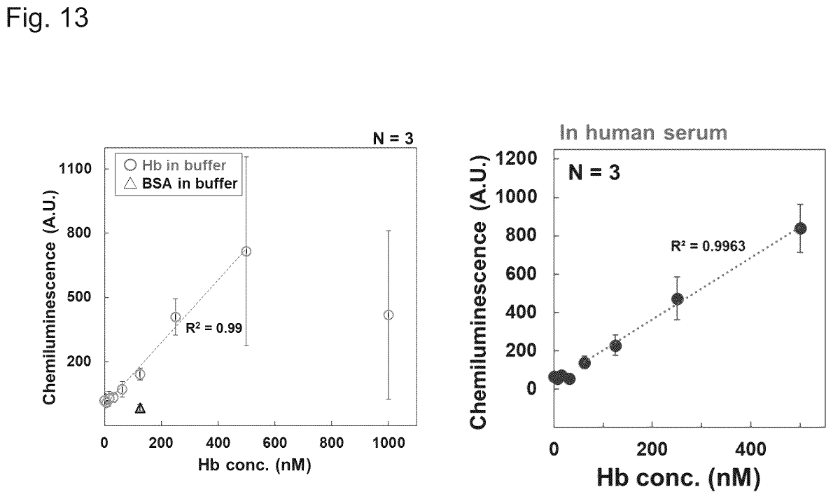

Hb in serum was detected by a low protein adsorption 96-well plate made of polystyrene. The result are shown in FIG. 13. An increase in chemiluminescence was confirmed in a concentration dependent manner of Hb similarly to the control (FIG. 13, left figure) in which Hb diluted with 10 mM P.P.B. (pH 6.0) was detected. From the results, it was suggested that Hb in serum can be detected. The linearity of chemiluminescence intensity was confirmed in the range of Hb concentration from 62.5 to 500 nM. The clinically desired detection range of Hb in blood is 1.1 to 3.1 mM. Accordingly, it was suggested that the clinically desired detection range of Hb in blood can be satisfied by diluting blood 1.0×10⁴ fold in the detection system of the invention.

### [Example 3: Preparation Example of Protein-Nucleic Acid Complex]

In this Example, a plurality of protein-nucleic acid complexes different from those prepared in the above Examples were prepared and the versatility of the preparation method for a complex using UdgX was verified.

In this Example, glucose dehydrogenase (GDH) was used as the protein domain of a complex and an aptamer TBA15 (5′-GGTTGGTGTGGTTGG-3′: SEQ ID NO: 8) which binds to thrombin was used as a nucleic acid. More specifically, an aptamer TBA15-GDH complex was tried to be prepared. First, a fusion protein (referred to as GDH-SpyCatvcher) having glucose dehydrogenase and SpyCatcher arranged in the order from the N terminal was prepared. Note that, the amino acid sequence of GDH-SpyCatcher is shown in SEQ ID NO: 9.

Subsequently, to prepare a UdgX-GDH complex, a SpyTag/SpyCatcher reaction was performed. Then, 2.5 μL of 20 μM UdgX-SpyTag (Example 2, SEQ ID NO: 2) and 2.5 μL of 20 μM GDH-SpyCatcher, each diluted with a 10 mM Sodium phosphate buffer (pH 6.0) comprising 10 mM KCl were mixed and incubated at 4 °C for 24 hours in a stand-still condition. The GDH-SpyCatcher used herein was produced using a recombinant E. coli and purified from a soluble fraction of the E. coli cells by His-tag purification. The GDH-SpyCatcher obtained was confirmed by SDS-PAGE analysis.

Finally, a TBA15-GDH complex was prepared as follows. That is, 20 μM UdgX-SpyTag and 20 μM GDH-SpyCatcher, each diluted with a 10 mM Sodium phosphate buffer (pH 6.0) comprising 10 mM KCl were mixed in the same amounts and incubated at 4 °C for 24 hours. To prepare a TBA15-GDH complex, a sequence having the UdgX recognition sequence, TBA15, and a fluorescent dye FAM in combination was used for a DNA binding reaction. A 10 UM UdgX-GDH complex and 10 μM TBA15 having the UdgX recognition sequence were mixed in a reaction buffer (10 mM Sodium phosphate buffer (pH 6.0) comprising 10 mM KCl) and incubated at 4 °C overnight. The obtained sample was subjected to SDS-PAGE analysis. The protein was confirmed by Coomassie BrilliantBlue (CBB) staining and the nucleic acid was confirmed by fluorescence derived from FAM.

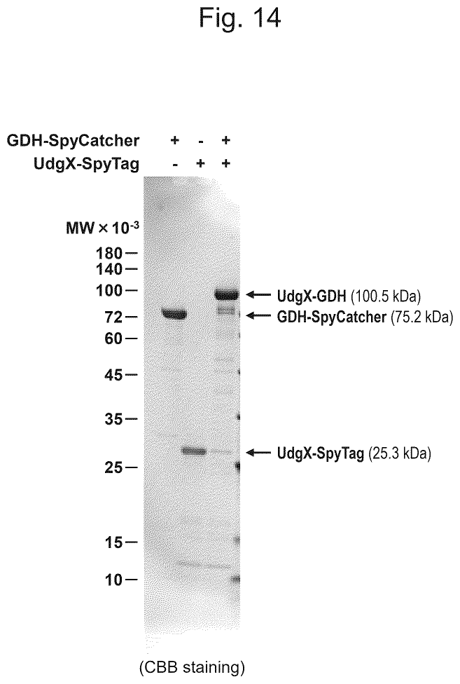

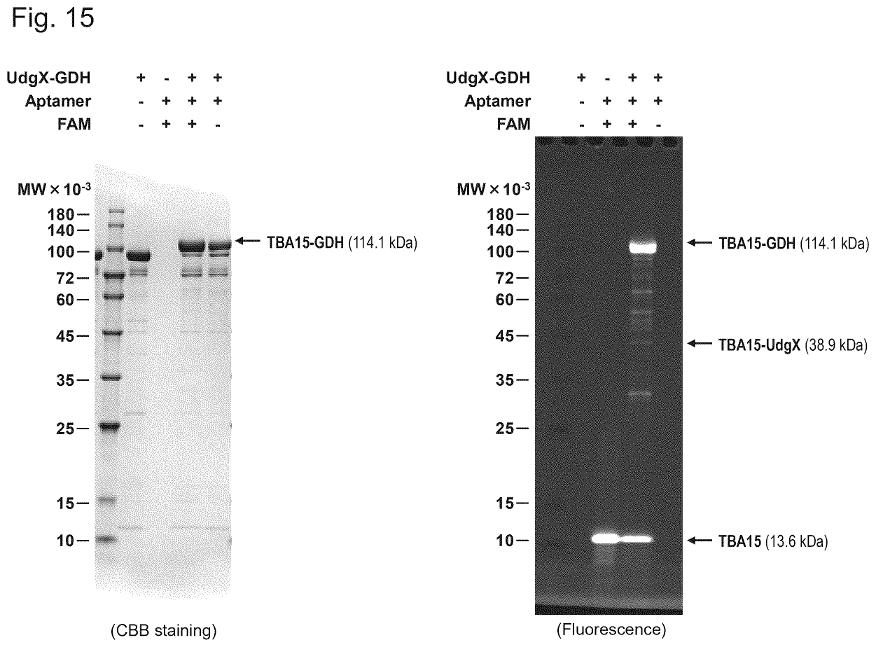

The results of SDS-PAGE analysis when a SpyTag/SpyCatcher reaction was performed using UdgX-SpyTag and GDH-SpyCatcher in the above Example are shown in FIG. 14. As shown in FIG. 14, a band was confirmed near a theoretical molecular weight (100.5 kDa) of the UdgX-GDH complex. From this, it was successfully confirmed that a UdgX-GDH complex was formed by a connection module, SpyTag/SpyCatcher system. The results of SDS-PAGE analysis and the results of fluorescence detection when the UdgX-GDH complex prepared and a TBA15 aptamer were reacted are shown in FIG. 15. As shown in FIG. 15, a band was confirmed near a theoretical molecular weight (114.1 kDa) of the complex formed by binding a TBA15 aptamer to a UdgX-GDH complex, it was successfully confirmed that a protein-nucleic acid complex using UdgX was formed.

In this Example, as another protein-nucleic acid complex using UdgX, a protein-nucleic acid complex was prepared using a UdgX-GOx complex prepared in Example 2 and the aforementioned TBA15 aptamer.

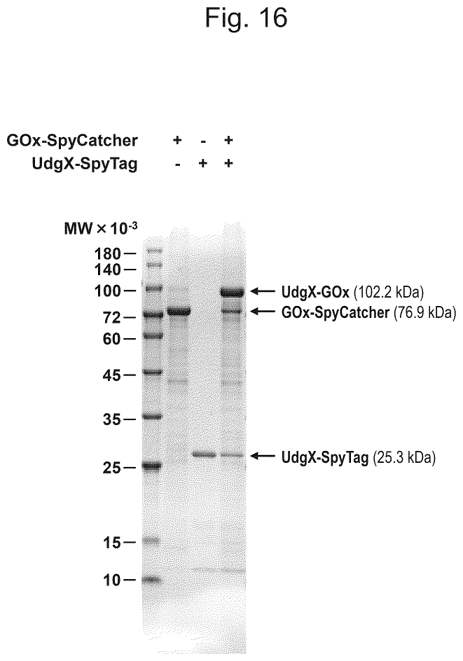

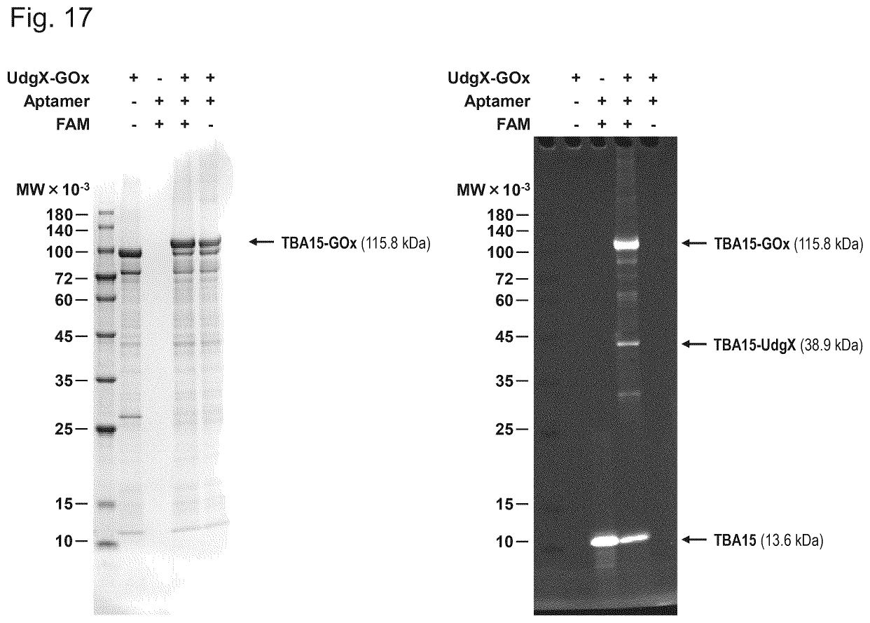

The results of the SDS-PAGE analysis when a SpyTag/SpyCatcher reaction was performed using UdgX-SpyTag and GOx-SpyCatcher are shown in FIG. 16. As shown in FIG. 16, a band was confirmed near a theoretical molecular weight (102.2 kDa) of the UdgX-GOx complex. From this, it was successfully confirmed that the UdgX-GOx complex was formed by a connection module, SpyTag/SpyCatcher system. Also, the results of the SDS-PAGE analysis and the results of fluorescence detection when the UdgX-GOx complex prepared and the TBA15 aptamer were reacted are shown in FIG. 17. As shown in FIG. 17, a band was confirmed near a theoretical molecular weight (115.8 kDa) of a complex prepared by binding the TBA15 aptamer to the UdgX-GOx complex. From this, it was successfully confirmed that a protein-nucleic acid complex using UdgX was formed.

Furthermore, in this Example, as another protein-nucleic acid complex using UdgX, a protein-nucleic acid complex was prepared using the UdgX-GDH complex prepared in this Example and the PEA3-01 aptamer prepared in Example 2.

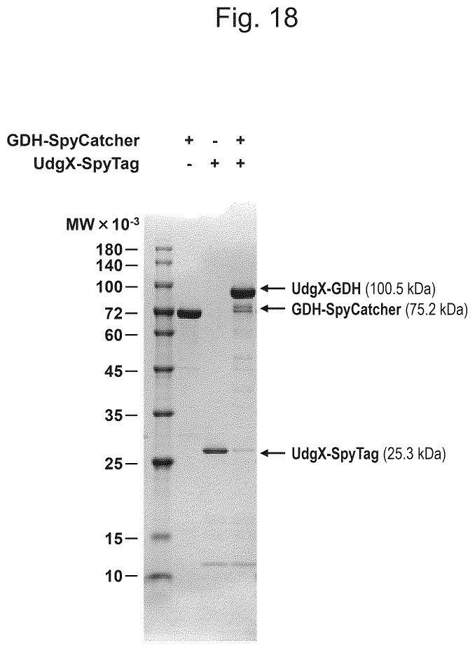

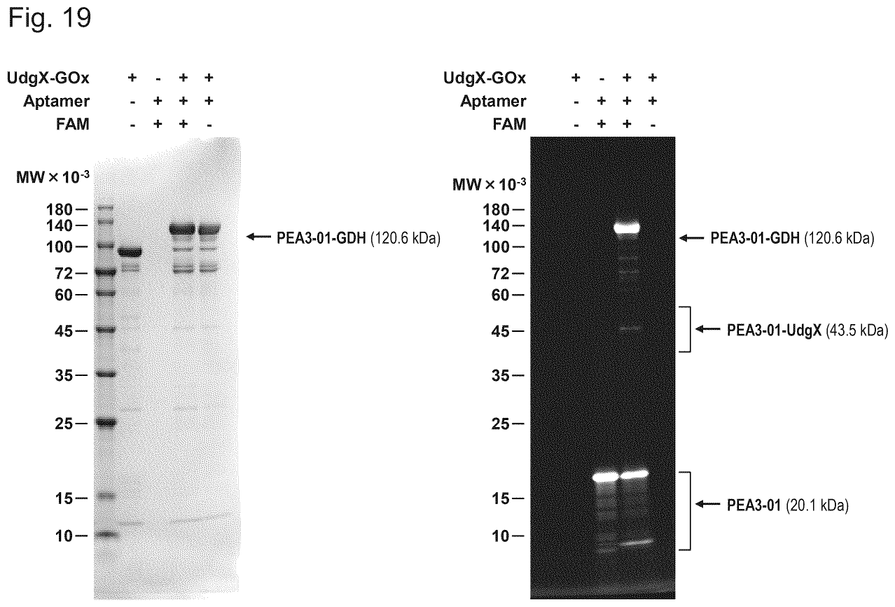

The results of the SDS-PAGE analysis when a SpyTag/SpyCatcher reaction was performed using UdgX-SpyTag and GDH-SpyCatcher were shown in FIG. 18. As shown in FIG. 18, a band was confirmed near a theoretical molecular weight (100.5 kDa) of the UdgX-GDH complex. From this, it was successfully confirmed that a UdgX-GDH complex was formed by a connection module, spyTag/SpyCatcher system. Also, the results of the SDS-PAGE analysis and the results of fluorescence detection when the UdgX-GDH complex prepared and the PEA3-01 aptamer were reacted are shown in FIG. 19. As shown in FIG. 19, a band was confirmed near a theoretical molecular weight (120.6 kDa) of a complex prepared by binding the PEA3-01 aptamer to a UdgX-GDH complex. From this, it was successfully confirmed that a protein-nucleic acid complex using UdgX was formed.

As mentioned in the above, it was demonstrated by this Example that 3 different types of protein-nucleic acid complexes can be formed by using UdgX. As a result, versatility of the method for preparing a protein-nucleic acid complex using UdgX was demonstrated.

All publications, patents and patent applications used in the specification are incorporated herein in their entirety by reference.

1. A protein-nucleic acid complex comprising a nucleic acid molecule having DNA comprising uracil, a uracil DNA glycosylase domain covalently bound to the nucleic acid molecule, and a protein domain covalently bound directly or indirectly to the uracil DNA glycosylase domain, wherein the uracil DNA glycosylase domain and the protein domain are covalently bound via a tag peptide and a tag capture peptide.

2. The protein-nucleic acid complex according to claim 1, wherein the uracil DNA glycosylase domain forms a fusion protein with the tag peptide or the tag capture peptide and the protein domain forms a fusion protein with the tag capture peptide or the tag peptide so that the uracil DNA glycosylase domain and the protein domain are covalently bound via the tag peptide and the tag capture peptide indirectly.

3. The protein-nucleic acid complex according to claim 1, wherein the nucleic acid molecule is a nucleic acid aptamer molecule which specifically binds to a predetermined substance or a molecule comprising a nucleotide sequence complementary to a predetermined nucleotide sequence.

4. The protein-nucleic acid complex according to claim 1, wherein the nucleic acid molecule is a chemically modified nucleic acid molecule.

5. A biosensing element comprising the protein-nucleic acid complex according to claim 1.

6. The biosensing element according to claim 5, wherein the biosensing element detects a substance to which the nucleic acid molecule comprised in the protein-nucleic acid complex specifically binds.

7. The biosensing element according to claim 5, wherein the biosensing element detects a substance to which the protein domain comprised in the protein-nucleic acid complex specifically binds.

8. The biosensing element according to claim 5, wherein the nucleic acid molecule comprised in the protein-nucleic acid complex is an aptamer molecule which increases peroxidase activity of a heme protein, and the protein domain comprised in the above protein-nucleic acid complex is an enzyme having oxidase activity which catalyzes an oxidation reaction to generate hydrogen peroxide.

9. The biosensing element according to claim 5, wherein the protein-nucleic acid complex is immobilized onto a substrate via chemical modification of the nucleic acid molecule comprised in the protein-nucleic acid complex.

10. A kit for detecting a substance, comprising
a protein-nucleic acid complex according claim 1, in which a protein domain is an enzyme having oxidase activity which catalyzes an oxidation reaction to generate hydrogen peroxide,
a first substrate as a substrate for the enzyme, and
a second substrate as a substrate for a substrate to be detected to which the nucleic acid molecule of the protein-nucleic acid complex binds and which has peroxidase activity.

11. The detection kit according to claim 10, wherein the enzyme and the first substrate are glucose oxidase and glucose, respectively; and the substance to be detected and the second substrate are hemoglobin and luminol, respectively.

12. The detection kit according to claim 10, wherein the enzyme is a fusion protein with an antibody to the substance to be detected.

13. A method for detecting a substance comprising the steps of:
mixing the detection kit according to claim 10 and a sample that possibly comprises a substance to be detected; and
measuring an enzyme reaction consuming the second substrate comprised in the detection kit, wherein
the substance to be detected in the sample is detected based on the enzyme reaction.

14. A fusion protein of a uracil DNA glycosylase domain and a tag peptide or tag capture peptide, wherein the fused protein is used for indirectly and covalently binding a nucleic acid molecule having DNA comprising uracil and a protein domain which is covalently bound to a tag capture peptide or a tag peptide.


SEQUENCE LISTING (9 sequences)

SEQ ID NO: 1 - DNA, 65 bp, synthetic construct
  misc_feature 1..65: note = Synthetic nucleic acid
  misc_feature 29: note = The 29th nucleotide represents uracil
  source 1..65: mol_type = other DNA
gggcgggttgggctgggtttctcaagtgtaggcatgcaagagctttttctacatctcagcattct

SEQ ID NO: 2 - DNA, 717 bp, synthetic construct
  misc_feature 1..717: note = Synthetic nucleic acid
  source 1..717: mol_type = other DNA
  CDS 1..717
atggcgggtgcgcaagatttcgtcccccacacggcggacctggccgaactcgctgccgcggcaggcgaatgccgaggctgcgggttgtatcgcgacgccacgcaggcggtcttcggcgcgggcgggcgctcggcacgcatcatgatgatcggcgagcagcccggtgacaaagaggacctggcgggcctgcccttcgtgggtcccgcgggccggctcctggaccgcgcgctggaagccgcggacatcgaccgcgatgccctctacgtcaccaacgcggtcaagcacttcaagttcacgcgggcagcgggaggcaaacgacgcatccacaagacccccagtcgtaccgaggtggtggcgtgccgtccctggctcatcgccgagatgacctccgtggagcccgatgtcgtggtgctgctcggcgcaaccgccgccaaggcactgctgggcaacgactttcgcgtcacgcagcatcgcggcgaggtcctgcacgtcgacgacgtaccgggagatccggcgctggtcgccaccgttcacccgtcgtctctgctgcgcggacccaaggaggaacgcgaatccgcgttcgcgggcctggtcgacgatctgcgtgtcgcagcagatgtcaggccaggtagtggtgcacatattgttatggttgatgcatataaaccgaccaaaaagcttgcggccgcactcgagcaccaccaccaccaccactga

SEQ ID NO: 3 - protein, 238 aa, synthetic construct
  REGION 1..238: note = Synthetic Construct
MAGAQDFVPHTADLAELAAAAGECRGCGLYRDATQAVFGAGGRSARIMMIGEQPGDKEDLAGLPFVGPAGRLLDRALEAADIDRDALYVTNAVKHFKFTRAAGGKRRIHKTPSRTEVVACRPWLIAEMTSVEPDVVVLLGATAAKALLGNDFRVTQHRGEVLHVDDVPGDPALVATVHPSSLLRGPKEERESAFAGLVDDLRVAADVRPGSGAHIVMVDAYKPTKKLAAALEHHHHHH

SEQ ID NO: 4 - DNA, 1026 bp, synthetic construct
  misc_feature 1..1026: note = Synthetic nucleic acid
  source 1..1026: mol_type = other DNA
  CDS 1..1026
atggcgggtgcgcaagatttcgtcccccacacggcggacctggccgaactcgctgccgcggcaggcgaatgccgaggctgcgggttgtatcgcgacgccacgcaggcggtcttcggcgcgggcgggcgctcggcacgcatcatgatgatcggcgagcagcccggtgacaaagaggacctggcgggcctgcccttcgtgggtcccgcgggccggctcctggaccgcgcgctggaagccgcggacatcgaccgcgatgccctctacgtcaccaacgcggtcaagcacttcaagttcacgcgggcagcgggaggcaaacgacgcatccacaagacccccagtcgtaccgaggtggtggcgtgccgtccctggctcatcgccgagatgacctccgtggagcccgatgtcgtggtgctgctcggcgcaaccgccgccaaggcactgctgggcaacgactttcgcgtcacgcagcatcgcggcgaggtcctgcacgtcgacgacgtaccgggagatccggcgctggtcgccaccgttcacccgtcgtctctgctgcgcggacccaaggaggaacgcgaatccgcgttcgcgggcctggtcgacgatctgcgtgtcgcagcagatgtcaggccaggtagtggtggtgcaatggttgataccctgagcggtctgagcagcgaacagggtcagagcggtgatatgaccattgaagaagatagcgcaacccacatcaaattcagcaaacgtgatgaagatggcaaagaactggcaggcgcaacaatggaactgcgtgatagcagtggtaaaaccattagcacctggattagtgatggtcaggtgaaagatttttatctgtatccgggtaaatataccttcgttgaaaccgcagcaccggatggttatgaagttgcaaccgcaattacctttaccgtgaatgaacaaggtcaggttaccgttaatggtaaagcaaccaaaggtgatgcacatattaagcttgcggccgcactcgagcaccaccaccaccaccactga

SEQ ID NO: 5 - protein, 341 aa, synthetic construct
  REGION 1..341: note = Synthetic Construct
MAGAQDFVPHTADLAELAAAAGECRGCGLYRDATQAVFGAGGRSARIMMIGEQPGDKEDLAGLPFVGPAGRLLDRALEAADIDRDALYVTNAVKHFKFTRAAGGKRRIHKTPSRTEVVACRPWLIAEMTSVEPDVVVLLGATAAKALLGNDFRVTQHRGEVLHVDDVPGDPALVATVHPSSLLRGPKEERESAFAGLVDDLRVAADVRPGSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIKLAAALEHHHHHH

SEQ ID NO: 6 - DNA, 24 bp, synthetic construct
  misc_feature 1..24: note = Synthetic nucleic acid
  misc_feature 9: note = The 9th nucleotide represents uracil
  source 1..24: mol_type = other DNA
ctcaagtgtaggcatgcaagagct

SEQ ID NO: 7 - protein, 708 aa, synthetic construct
  REGION 1..708: note = a fusion protein
MNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQGSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIHHHHHH

SEQ ID NO: 8 - DNA, 15 bp, synthetic construct
  source 1..15: mol_type = other DNA
  misc_feature 1..15: note = Synthetic nucleic acid
ggttggtgtggttgg

SEQ ID NO: 9 - protein, 698 aa, synthetic construct
  REGION 1..698: note = a fusion protein
MNTTTYDYIVVGGGTSGLVVANRLSENPDVSVLLLEAGASVFNNPDVTNANGYGLAFGSAIDWQYQSINQSYAGGKQQVLRAGKALGGTSTINGMAYTRAEDVQIDVWQKLGNEGWTWKDLLPYYLKSENLTAPTSSQVAAGAAYNPACNGKEGPLKVGWSGSLASGNLSVALNRTFQAAGVPWVEDVNCGKMRGFNIYPSTLDVDLNVREDAARAYYFPYDDRKNLHLLENTTANRLFWKNGSAEEAIADGVEITSADGKVTRVHAKKEVIISAGALRSPLILELSGVGNPTILKKNNITPRVDLPTVGENLQDQFNNGMAGEGYGVLAGASTVTYPSISDVFGNETDSIVASLRSQLSDYAAATVKVSNGHMKQEDLERLYQLQFDLIVKDKVPIAEILFHPGGGNAVSSEFWGLLPFARGNIHISSNDPTAPAAINPNYFMFEWDGKSQAGIAKYIRKILRSAPLNKLIAKETKPGLSEIPATAADEKWVEWLKANYRSNFHPVGTAAMMPRSIGGVVDNRLRVYGTSNVRVVDASVLPFQVCGHLVSTLYAVAERASDLIKEDAKSAGSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHILEHHHHHH